(19) United States
(12) Patent Application Publication
(10) Pub. No.: US 2013/0251702 A1
(43) Pub. Date: Sep. 26, 2013

(54) METHOD FOR SCREENING COMPOUNDS FOR TREATING SEPSIS TARGETING NOD2 SIGNALLING PATHWAY AND COMPOSITION FOR TREATING SEPSIS COMPRISING NOD2 SIGNALLING PATHWAY INHIBITORS

(71) Applicants: Doo Hyun CHUNG, Seoul (KR); Sae Jin OH, Seoul (KR); Ji Hyung KIM, Seoul (KR)

(72) Inventors: Doo Hyun CHUNG, Seoul (KR); Sae Jin OH, Seoul (KR); Ji Hyung KIM, Seoul (KR)

(73) Assignee: SNU R&DB FOUNDATION, Seoul (KR)

(21) Appl. No.: 13/848,286

(22) Filed: Mar. 21, 2013

(30) Foreign Application Priority Data
Mar. 21, 2012 (KR) 2012-0028748

(51) Int. Cl. C12Q 1/48; G01N 33/74; A61K 31/4439; C12Q 1/68

(52) U.S. Cl. CPC C12Q 1/485; C12Q 1/68; G01N 33/74; A61K 31/4439
USPC 424/130.1; 435/15; 435/6.13; 436/501; 435/7.1; 435/6.12; 435/7.92; 514/341; 514/252.18; 514/252.19; 514/275; 514/234.5; 514/266.4; 514/266.24; 514/266.22; 514/318; 514/346; 514/414; 514/338; 514/300; 514/291; 514/253.07; 514/44 A; 514/1.4

(57) ABSTRACT

Methods for screening compounds for treating sepsis are disclosed. The present methods and compositions are targeting NOD2 mediated signaling pathway and the agents identified by the present methods are qualified as drug candidates for clinical development. Further Methods and composition for treating sepsis are disclosed.

### CROSS-REFERENCE TO RELATED APPLICATIONS

This application claims the benefit of Korean Patent Application NO. 2012-0028748 filed Mar. 21, 2012 in the Korean Intellectual Property Office, disclosure of which is incorporated herein by reference.

### BACKGROUND OF INVENTION

1. Field of the Invention

The present disclosure generally relates to compositions and methods to treat sepsis targeting NOD2 signaling pathway and screening method for identifying a therapeutic agent for sepsis.

2. Description of the Related Art

Sepsis is a complex dysregulated inflammatory response in infection, which causes multiple organ dysfunction and coagulopathy, often resulting in death. The fatality is about 70% and worldwide over 1500 people die of septic shock each day. However effective therapies for sepsis have yet to be developed. Xigris (Drotrecogin alfa) from Eli Lilly Company is the only approved drug for sepsis. Xigris is a recombinant form of human activated protein C that has anti-thrombotic and anti-inflammatory properties. In 2001, the FDA approved the use of Xigris in patients with severe sepsis and at a high risk of death. The clinical trial in pediatric patients with severe blood infection resulted in a failure due to the severe side effect of cerebral hemorrhage.

Also Critical Care Medicine reported in 2009 that severe hemorrhage had occurred and the fatality had increased in 73 patients treated with Xigris.

Initially excessive production and release of cytokines can initiate widespread tissue injury which can often lead to organ damages in sepsis. Thus, much of the research has been focused on the inhibition of proinflammatory materials such as cytokines. However, there has been reports that outcome of cytokine therapies were not effective and may worsen the prognosis due to its stimulatory effect on the bacterial growth. (O'Reilly, M et al. 1999. Shock 12, 411-20; F Fisher, C. J. et al 1996. N. Engl. J. Med. 334, 1697-702). Recently it has also been reported that the initial excessive innate immunological reaction led to the immunoparalysis and the patients die of immunoparalysis caused by primary or secondary infection with pathogens (Docke, W. D. et al. 1999. Nature Med. 3: 678-81). These results indicate that the treatment based on initial inhibition of inflammatory cytokines has its limit.

US Patent Application Publication No. 2008/0311112 discloses use of toll-like receptor-9 agonists, toll-like receptor-4 antagonists, and/or nuclear oligomerization domain-2 agonists for the treatment or prevention of toll-like receptor-4-associated disorders. However it does not disclose an effective therapy based on reducing the initial immune reaction and also effectively eliminating pathogens.

Therefore there exist needs to develop a safe and effective new drug that can inhibit the excessive immune reaction and is also effective in eliminating pathogenic microorganisms.

### SUMMARY OF THE INVENTION

The present disclosure is to provide a therapeutic composition to treat anxiety disorder such as PTSD and phobia with good and extended medicinal effect.

In one aspect, the present disclosure provides a method of screening a therapeutic agent for sepsis that regulates NOD2 mediated signaling pathway. In one embodiment, the method comprises (i) providing a RIP2 (RICK) protein; (ii) contacting the RIP2 protein with a test substance wherein the substance is expected to inhibit the phosphorylation or kinase activity of the RIP2 protein; and (iii) selecting a test substance as a candidate that decreases the phosphorylation level or the kinase activity of the RIP2 protein in comparison to a negative control that is not contacted with a test substance. In one embodiment, RIP2 protein is provided as a cell that endogenously or exogenously express RIP2 protein such as a neutrophil, an epithelial cell, 293T, 293 or 293A or any combination thereof. The method may further comprise selecting a test substance that increases the expression of a CD55 and/or decreases the level of C5a.

Also provided in the present disclosure is a method of screening a therapeutic agent for sepsis based on the inhibition of protein-protein interaction involved in NOD2 mediated signaling pathway. In one embodiment, it comprises (i) providing either a NOD2 or the CARD region of a NOD2 protein, and either a RIP2 or the CARD region of a RIP2 protein; (ii) contacting the proteins with a test substance wherein the substance is expected to interfere with the interaction of the NOD2 or the CARD region of the NOD2 with the RIP2 or the CARD region of the RIP2 protein; and (iii) detecting the interaction of (ii) and selecting a test substance as a candidate that interfere with the interaction. In one embodiments, the NOD2, RIP2 proteins and their CARD regions are provided as cells that endogenously and/or exogenously express them, which for example includes a dendritic cell, a neutrophil, an epithelial cell, 293T, 293 or 293A or any combination thereof. The method may further comprise selecting a test substance that increases the expression of a CD55 and/or decreases the level of C5a.

In other aspect, the present disclosure provides a pharmaceutical composition for treating sepsis comprising an inhibitor of the kinase activity of a RIP2 protein and/or an inhibitor of the phosphorylation of a RIP2 protein. In one embodiment, the inhibitor is selected from the group consisting of Imatinib, Dasatinib, Niolotinib, Gefitinib, Erlotinib, Afatinib, Dacomitinib, Crizotinib, Sorafenib, Sunitinib, Pazopanib, Axitinib, Lapatinib, Vemurafenib, Everolimus, Temsirolimus, Dovitinib and SB203580.

In other aspect, the present disclosure provides a pharmaceutical composition for treating sepsis comprising an NOD2 and/or RIP2 inhibitor, wherein the inhibitor suppress the expression and/or activity of the NOD2 or RIP2, which includes small molecules, antibodies, antisense oligonucleotides, siRNAs, shRNAs miRNAs and polypeptides.

Also provided in the present disclosure is a method of treating sepsis comprising administering to a subject in need thereof an effective amount of an inhibitor of the kinase activity of a RIP2 protein and/or an inhibitor of the phosphorylation of a RIP2 protein. Kinase inhibitors which may be used include Imatinib, Dasatinib, Niolotinib, Gefitinib, Erlotinib, Afatinib, Dacomitinib, Crizotinib, Sorafenib, Sunitinib, Pazopanib, Axitinib, Lapatinib, Vemurafenib, Everolimus, Temsirolimus, Dovitinib and SB203580.

In still other aspect, the present disclosure provides a method of treating sepsis comprising administering to a subject in need thereof an effective amount of one or more NOD2 inhibitors and/or RIP2 inhibitors, which may include small molecules, antibodies, antisense oligonucleotides, siRNAs, shRNAs miRNAs and polypeptides.

In still other aspect, the present disclosure provides a method of treating sepsis comprising administering to a subject in need thereof an effective amount of one or more inhibitors that interfere with RIP2 and NOD2 interaction. In one embodiment the interaction is accomplished through a CARD region present on the RIP2 and NOD2 protein.

The foregoing summary is illustrative only and is not intended to be in any way limiting. Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1A to 1F shows attenuated CLP induced sepsis by suppressing C5a generation in Nucleotide-binding oligomerization domain (Nod2)^{-/-} mice. (A) Serum and peritoneal C5a and C3a levels were evaluated in WT and Nod2^{-/-} mice 24 h after CLP. (B) The percentages of surviving mice were estimated during CLP-induced sepsis (P<0.001, log-rank test, n=5 per group; WT vs. Nod2^{-/-} mice, mice injected with recombinant C5a vs. PBS) (C) The peritoneal cells obtained from WT, Nod2^{-/-}, Nod2^{-/-} mice injected with recombinant C5a 24 h after CLP were incubated with LPS or PBS for 6 h and cytokine levels were measured. The ratios of individual cytokines produced were determined by estimating cytokine levels in culture supernatant fractions of LPS vs. PBS. (D) Serum D-dimer levels were measured in WT, Nod2^{-/-}, Nod2^{-/-} mice injected with recombinant C5a 24 h after CLP. (E) Phagocytosis activity of the peritoneal cells obtained from mice 24 h after CLP was determined by measuring mean fluorescence intensity (MFI) of intracellular FITC-conjugated E. coli after 15 min incubation. (F) Bacterial CFUs were counted using blood and liver homogenates obtained from mice 24 h after CLP. Results shown are representative of three independent experiments. *P<0.05, **P<0.01, ***P<0.001 for WT B6 vs. Nod2^{-/-} mice

FIG. 2A to 2E Nucleotide-binding oligomerization domain (NOD)₂-mediated signals induce IL-1β and IL-10 production by Ly-6G⁺ peritoneal cells during cecal ligation and puncture (CLP)-induced sepsis. (A) Serum and peritoneal IL-6, IFN-γ, TNF-α, IL-1β, and IL-10 levels were measured in WT and Nod2^{-/-} mice 24 h after CLP. (B) Peritoneal cells from WT or Nod2^{-/-} mice with CLP were incubated with or without MDP for 24 h and amounts of IL-1β and IL-10 in culture supernatant were measured. (C) Serum and peritoneal IL-1β and IL-10 levels were estimated in WT and Nod2^{-/-} mice 4, 12, and 24 h after CLP using ELISA. (D) The NOD2 expression pattern was estimated in sorted F4/80⁻Ly-6G⁺ and F4/80⁺Ly-6G⁻ peritoneal cells of WT mice 4, 12, and 24 h after CLP using real-time PCR. (E) IL-1β and IL-10 transcript levels were evaluated in sorted F4/80⁻Ly-6G⁺ and F4/80⁺Ly-6G⁻ peritoneal cells from WT and Nod2^{-/-} mice 4, 12, and 24 h after CLP. Results shown are representative of three independent experiments. *P<0.05, **P<0.01, ***P<0.001 for WT B6 vs. Nod2^{-/-} mice.

FIG. 3A to 3D shows IL-1β dependent IL-10 production mediated via nucleotide-binding oligomerization domain (NOD) 2 enhances C5a generation during cecal ligation and puncture (CLP)-induced sepsis. (A) IL-1β and IL-10 receptor expression was estimated on peritoneal immune cells from WT B6 mice 4 h and 24 h after CLP. (B) Serum and peritoneal C5a and C3a levels were measured in WT and Nod2^{-/-} mice injected with recombinant IL-1β or IL-10 after CLP. (C) The peritoneal cells obtained from WT, Nod2^{-/-}, Nod2^{-/-} mice injected with recombinant IL-1β or IL-10 were incubated with LPS or PBS for 6 h and cytokine levels were measured. The ratios of individual cytokines produced were determined by estimating cytokine levels in culture supernatant fractions of LPS vs. PBS. (D) The survival percentages of WT and Nod2^{-/-} mice injected with recombinant IL-1β or IL-10 were measured during CLP-induced sepsis (P<0.05, log-rank test, n=4 per group; WT or Nod2^{-/-} vs. WT or Nod2^{-/-} mice treated with recombinant IL-1β or IL-10). Results shown are representative of three independent experiments. *P<0.05, **P<0.01, ***P<0.001.

FIG. 4A to 4G shows NOD2-mediated IL-1β-dependent IL-10 production by Ly6-G⁺ granulocytes enhances C5a generation during sepsis. (A) Serum and peritoneal IL-10 levels were measured in WT, Nod2^{-/-}, and Nod2^{-/-} mice injected with recombinant IL-1β after CLP. (B) Serum and peritoneal C5a levels were measured in WT B6 mice injected with anti-IL-1β receptor or isotype-matched control mAb 24 after CLP. (C and D) Serum and peritoneal C5a and C3a levels were measured in WT, Il-10^{-/-}, Il-10^{-/-} mice injected with recombinant IL-1β after CLP. (E) Recombinant IL-1β or IL-10 was injected into Nod2^{-/-} mice and recombinant IL-1β was injected into IL-10^{-/-} mice after CLP. Peritoneal cells obtained from these mice were incubated with or without LPS and cytokine levels were measured. The ratios of individual cytokines (stimulated with LPS/un-stimulated) were estimated. (F and G) Recombinant IL-1β or IL-10 was injected into WT and Nod2^{-/-} mice after CLP. (F) The phagocytotic activity of peritoneal cells from these mice was determined by measuring intracellular fluorescence intensity after incubating the peritoneal cells with FITC-conjugated E. coli. (F) Bacterial CFUs were estimated using blood and liver homogenates obtained from WT and Nod2^{-/-} mice 24 h after CLP. Results shown are representative of three independent experiments. *P <0.05, **P<0.01, ***P<0.001.

FIG. 5A to 5E shows IL-1β-dependent IL-10 production mediated through nucleotide-binding oligomerization domain (NOD) 2 enhances C5a generation by suppressing CD55 expression on Ly6-G⁺ cells during sepsis. (A) CD55 and CR1/2 expression was estimated on gated Ly6-G⁺ peritoneal cells from WT, Nod2^{-/-}, Nod2^{-/-} mice injected with recombinant IL-1β or IL-10 24 h after CLP. (B) CD55 expression was estimated on gated Ly6-G⁺ peritoneal cells from Il-10^{-/-} or Il-10^{-/-} mice injected with recombinant IL-1β 24 h after CLP. (C) To block IL-10 receptor engagement in vivo, anti-IL10 receptor mAbs were i.p. injected into WT B6 and Nod2^{-/-} mice given recombinant IL-10 during CLP-induced sepsis. CD55 expression was evaluated on gated Ly6-G⁺ peritoneal granulocytes from these mice 24 h after CLP. (D) Peritoneal cells were obtained from WT and Nod2^{-/-} mice with CLP and cultured for 24 h. Then, amounts of C5a were measured in culture media. (E and F) To evaluate CD55 effect on C5a generation in vivo, WT B6 (E), or Nod2^{-/-} mice given recombinant IL-10 (F) were i.p. injected with soluble CD55 after CLP. Serum and peritoneal C5a levels and the survival percentages of these mice were measured during CLP-induced sepsis (P<0.05, log-rank test, n=8 per group; WT or Nod2^{-/-} mice injected with recombinant IL-10 vs. WT or Nod2^{-/-} mice injected with recombinant IL-10 and soluble CD55). Results shown are representative of three independent experiments. *P<0.05, **P<0.01, ***P<0.001.

FIG. 6A to 6E shows SB203580, an inhibitor for RIP2 downstream of NOD2, attenuates CLP-induced sepsis. (A) The peritoneal immune cells of WT mice were cultured with SB203580 and/or MDP for 24 hs and amounts of IL-1β and IL-10 were measured in culture fraction. (B) The molecules related to NOD2-mediated signal transduction were blotted using peritoneal cells obtained from WT and Nod2^{-/-} mice 4 and 12 h after CLP. (C) Serum and peritoneal IL-1β and IL-10 levels were estimated in WT and WT mice injected with SB203580 4, 8, and 24 h after CLP using ELISA. (D) The levels of CD55 expression on Ly6-G⁺cells from WT and WT mice injected with SB203580 were measured 4, 8, and 24 h after CLP. (E) The percentages of surviving mice were estimated during CLP-induced sepsis (P<0.001, log-rank test, n=5 per group; WT mice injected with SB203580 vs. PBS).

FIG. 7 represents a diagram showing (1) RIP2 (NP_{—}003812.1) protein from human and (B) regions of NOD2 protein (BAJ13470.1).

### DETAILED DESCRIPTION OF THE EMBODIMENTS

Nucleotide-binding oligomerization domain (NOD) 2 is an intracellular pattern recognition receptor and plays a defensive role in bacterial infection by sensing bacterial peptidoglycans. C5a is a crucial complement product that regulates immune responses during sepsis. However, functional role of NOD2 and its relationship with C5a in sepsis have not been known. The present invention is based on a discovery that NOD2-mediated signal increases C5a level by suppressing CD55 expression on Ly6-G⁺ granulocytes via IL-1 β-dependent or -independent IL-10 production by Ly6-G⁺ granulocytes, thereby aggravating polymicrobial sepsis. Further the suppression of NOD2 mediated signals can lead to the blocked or reduced cytokine production during sepsis, which in turn suppress the expression of C5a, an etiologic agent of sepsis, by regulating the factors that affect the production of C5a, thereby preventing or treating sepsis.

NOD2 is a member of a family called NLR (Nod Like Receptors) which recognizes pathogen associated molecular patterns (PAMP) and innate immune activity. It comprises a region called CARD (Caspase Activation and Recruitment Domain) at its N-terminus. Upon activation, NOD2 forms an oligomer and interacts with RIP2 (nuclear factor (NF)-kB-activating kinase receptor-interacting protein 2, which also contains CARD domain. The CARD-CARD interaction now initiates transduction of series of signals, which in turn activates NF-κb leading to the cytokine secretion (Davis, K. M., Nakamura, S. & Weiser, J. N. Nod2 sensing of lysozyme-digested peptidoglycan promotes macrophage recruitment and clearance of S. pneumoniae colonization in mice. J Clin Invest 121, 3666-3676 (2011))

Based on the discovery reported herein, one aspect of the present disclosure relates to a screening method, for identifying agents useful for treating and/or preventing sepsis or symptoms associated therewith based on the regulation of NOD2 signaling pathway. The agents identified by the present methods are qualified as drug candidates for clinical development.

Particularly, the present disclosure relate to a method of screening a therapeutic agent for treating sepsis by suppressing the signal transduction through NOD2 pathway, where the agents may suppress the expression and/or activity of NOD2, or the expression and/or activities of proteins involved in the downstream of NOD2 pathway, for example RIP2, and/or the interaction between NOD2 and RIP2.

In one embodiment, the present methods relate to a method of screening, wherein the expression of RIP2 protein, phosphorylation and/or kinase activity thereof is regulated. The method comprises the steps of: (i) providing a RIP2 or RICK protein; contacting the RIP2 protein with a test substance wherein the substance is expected to inhibit the expression, the phosphorylation or kinase activity of the RIP2 protein; and selecting a test substance as a candidate that decreases or reduce the expression, phosphorylation level or the kinase activity of the RIP2 protein in comparison to a negative control that is not contacted with a test substance.

As used herein, the term “regulating expression and/or activity” generally refers to any process that functions to control or modulate the quantity or activity (functionality) of a cellular component. Static regulation maintains expression and/or activity at some given level. Upregulation refers to a relative increase in expression and/or activity. Accordingly downregulation refers to a relative decrease in expression and/or activity. In the present invention, regulation is preferably the downregulation of a cellular component, particularly C5a. Downregulation is synonymous with inhibition of a given cellular component's activity.

Sepsis is a disease characterized by an overwhelming systemic inflammatory response to infection. Bacterial sepsis is a complex systemic inflammatory syndrome caused by aggressive bacterial infection such as Enterococcus spp., Staphylococcus spp., Streptococcus spp, Enterobacteriacae family, Providencia spp. and Pseudomonas spp.in the blood. Sepsis usually begins with tremor, fever, falling blood pressure (septic shock), rapid breathing, rapid heart rate, and skin lesions. Within hours, sepsis may cause spontaneous clotting in blood vessels, severe hypotension, multiple organ failure, shock, and eventually death. Typically, these symptoms are caused by the excessive or uncontrolled activation of host defense mechanisms such as cytokines, leukocytes, and complement. In one embodiment, the sepsis is caused by polymicrobial infection.

As used herein, the terms “treat,” “treatment,” and “treating” include alleviating, abating or ameliorating at least one symptom of a disease or condition, and/or reducing severity, progression and/or duration thereof, and/or preventing additional symptoms, and includes prophylactic and/or therapeutic measures.

RIP2 proteins from various sources and sequences in the art may be used for the present disclosure as long as it contains a kinases activity. The sequence of RIP2 is known in the art, for example as NCBI reference NO. NP_{—}003812.1 In one embodiment, a full or partial length of RIP2 can be used.

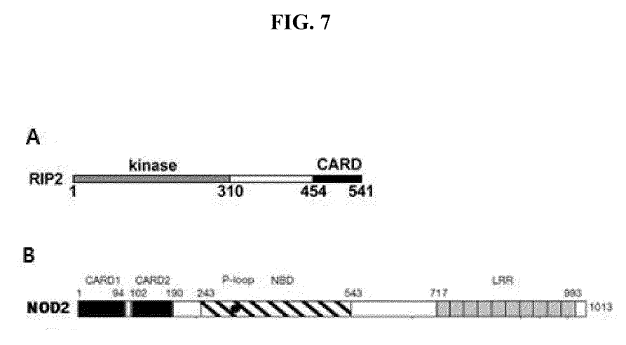

In one embodiment, full or partial RIP2 protein containing phosphorylation sites or exerting kinase activity as disclosed in FIG. 7 may be used.

Further if desired, RIP2 proteins from various origin may be utilized for the present methods. For example, RIP2 protein from mammal, particularly from human or mouse may be used, the sequences of which are known as NP_{—}003812.1 in human and NP 620402.1 in mouse.

Further RIP2 proteins and any functionally equivalent thereof may be used for the present invention. Also RIP2 proteins with some variations in sequence such as deletion, substitution, and addition may be used.

The RIP2 protein of the present disclosure may be prepared by methods known in the art. In one embodiment, recombinant technologies are used, in which for example a plasmid comprising a gene encoding RIP2 protein are introduced into prokaryotic or eukaryotic cells such as insect cells or mammalian cells for overexpression. The proteins are then extracted and purified or unpurified before use. Plasmids for cloning are known in the art and include but is not limited to vectors such as pET28b (Novagen, USA).

Further Nucleic acids encoding a protein used for the screening may be transcribed and/or translated in vitro and processed further before being used for the present methods. In one embodiment, the in vitro translated proteins may be further processed by centrifugation to remove the cell debris followed by purification by precipitation, dialysis, and column chromatography, the example of which includes ion exchange chromatography, gel-permeation chromatography, HPLC (high performance liquid chromatography), reverse phase HPLC, preparative SDS-PAGE, affinity column chromatography. The affinity column chromatography may be prepared using antibody against a protein of interest.

The proteins prepared as above may be used in vitro to identify agents useful for treating and/or preventing sepsis in the presence or the absence of a test substance. After contacting the protein with a test compound(s), the kinase activity of RIP2 protein may be measured/detected by methods known in the art. By way of example, in one embodiment, the detection methods include one using an antibody that specifically recognizes the phosphorylated form of a protein in protein blot assays. Such antibodies specific for phosphorylated or unphosphorylated RIP2 are commercially available from Cell signaling Inc. and Thermopierce Inc. With such antibodies the expression level and/or the level of phosphorylation can be detected and/or measured. The test agents that reduced the kinase activity or the level of phosphorylation in comparison to a negative control that does not treated with a test substance may be identified as a candidate for treating and/or preventing sepsis. The protein employed may be labeled for detection with labeling agent such as biotin, fluorescent compounds, and radioactive isotope using the methods known in the art or kits commercially available kits. The labeled products can be detected with a suitable reader in consideration of the label used and/or sensitivity desired and the like.

In other embodiment, RIP2 proteins are provided as a cell that endogenously or exogenously expressing the protein. For example, mammalian cells are prepared to express the protein of interest such as RIP2 through a transient or stable transfection or cells that endogenously express the protein of interest may be used. Cells endogenously expressing RIP2 may include but is not limited to, macrophages, dendritic cells, neutrophils and epithelial cells, which may be obtained from various organs for example such as peritoneal cavity of a mouse. The cells obtained may be cultured in a cell culture dish and treated with a test substance for a certain period time in a suitable medium, from which the whole proteins are extracted and tested/detected for kinase activity of RIP2 protein. Candidate substances useful for treating or preventing sepsis are selected based on the comparison with a negative control that does not contacted with a test substance. Alternatively established cell lines may be used, in which case the cells are transfected with a plasmid expressing RIP2. The example of such cells include but is not limited to 293, 293T or 293A (Graham F L, Smiley J, Russell W C, Nairn R (July 1977). “Characteristics of a human cell line transformed by DNA from human adenovirus type 5”. J. Gen. Virol. 36 (1): 59-74; and Louis N, Evelegh C, Graham F L (July 1997). “Cloning and sequencing of the cellular-viral junctions from the human adenovirus type 5 transformed 293 cell line”. Virology 233 (2): 423-9).

Further it has been found in the present disclosure that many of the symptoms of sepsis are resulted from CSa, the expression of which are affected by IL-1beta, IL-10 cytokines, the expression of which is regulated by NOD2. Such regulation of the production of complements by modulating the secretion of cytokines is possible through the regulation of CD55 in Ly6G⁺ cells (neutrophil) which is a negative regulator of CSa. As such, suppression of NOD2 mediated pathway can lead to the reduction in cytokines which results in the modulation of a protein that negatively regulates the etiologic protein of sepsis, C5a, thereby alleviating sepsis. In this perspective, the present methods may include a step of selecting a test substance that increases the expression of CD55 and/or decrease the expression of C5a in vivo in addition and/or in replace of identifying a candidate based on the regulation of RIP2 activity or phosphorylation thereof. The change in the level of C5a or production thereof after the development of sepsis can be detected in serum or in peritoneal cavity via ELISA (Enzyme Linked Immunosorbent Assay).

Based on the disclosure of the present invention that NOD2 mediated pathway is involved in the development of sepsis and interfering with the pathway would lead to the reduction in the level of etiologic agents, other aspect of the present invention is directed to a screening method for identifying an agent useful for treating or preventing sepsis by interfering the interactions of proteins involved in NOD2 pathway, particularly NOD2 and RIP2. In one embodiment the present method includes the steps of: (i) providing either a NOD2 or the CARD region of a NOD2 protein, and either a RIP2 or the CARD region of a RIP2 protein; (ii) contacting the proteins with a test substance wherein the substance is expected to inhibit an interaction of the NOD2 or the CARD region of the NOD2 with the RIP2 or the CARD region of the RIP2 protein; and (iii) detecting the interaction of (ii) and selecting a test substance as a candidate that decreases/interferes with the interaction.

The sequence of NOD2 and RIP2 proteins and their CARD regions are depicted in FIG. 7 but are not limited thereto and various other sequences that fulfill the present purpose may be used. In one embodiment, sequences originated from mammal particularly mouse or human are used. RIP2 sequences which may be used for the present methods are as described above, and CARD region of which is located from amino acid residues 454 to 541. NOD2 sequences from various origins are known in the art and full or partial length of NOD2, or the surrounding regions including NOD2 may be used. In one embodiment, NOD2 originated from human is used and disclosed as GenBank No. BAJ13470.1 wherein CARD region is from amino acid residues 1 to 91 and 102 to 190. It is understood that the specific location of CARD region may vary depending on the origin and the sequence variations even found in one species. Example of NOD2 sequence from mouse is disclosed as NP_{—}665856.2.

The effect of a test substance on the protein-protein interaction may be detected by a variety of methods known in the art. The example includes but is not limited to yeast two hybrid method, confocal microscopy, co-immunoprecipitation, surface plasmon resonance (SPR) and spectroscopy. Reference may be found in Berggard et al., (2007) “Methods for the detection and analysis of protein-protein interactions”, PROTEOMICS Vo17: pp 2833-2842. The interaction as used herein refers to contacting or binding among proteins, and includes interaction through covalent or non-covalent binding. The substance that resulted in a change and/or decrease in the interaction is selected as a candidate for treating or preventing sepsis.

The interference by a test substance of the interaction of NOD2 with RIP2 protein may be detected directly or indirectly. The indirect interaction may be detected by the effect on the expression of C5a and CD55. The detection of protein-protein interaction may be performed by the methods known in the art. Non limiting examples to detect a complex formation includes western blot, ELISA, Radial immunodiffusion assay, immunohistochemistry, immunoprecipitation method, complement fixation test, FACS or protein chip based assay. Test substance that reduces or decreases the expression of C5a is selected as a candidate.

Further NOD2 or RIP2 protein of the present methods may also be provided as a cell expressing the protein as described above. For example, 293T cells can be transfected with a plasmid expressing full or partial length of NOD2 and/or RIP2, followed by treatment with a test substance. Then candidates are selected based on a comparison with a negative control which does not treated with a test substance, wherein the decrease/reduction/interference in the interaction in cells treated with a test substance qualifies the test substance as candidate.

The amount of proteins used, types of cells and the amount and types of test substances may vary depending on the particular process, test substance employed, which can be appropriately selected by a skilled person in the art without undue burden. The substance that causes the reduction/suppression/inhibition in the activity of RIP2 kinases or its phosphorylation is selected as a candidate. The level of reduction/suppression/inhibition means the level not more than about 99%, about 95% about 90%, about 85%, about 80%, about 75%, about 70%, about 65%, about 60%, about 55%, about 50%, about 45%, about 40%, about 30%, about 20% in comparison to that of negative control, but is not limited thereto.

The term “test substance” “test agents” or “test compound” refers generally to a material that is expected to decrease, reduce, suppress or inhibit the kinase activity of RIP2 or its phosphorylation or to interfere the interaction between RIP2 and NOD2, which include small molecules, high molecular weight molecules, mixture of compounds such as natural extracts or cell or tissue culture products, biological material such as proteins, antibodies, peptides, DNA, RNA, antisense oligonucleotides, RNAi, aptamer, RNAzymes and DNAzymes, or glucose and lipids, but is not limited thereto. The test substances may be polypeptides having amino acid residues of below 20, particularly 6, 10, 12, 20 aa or above 20 such as 50aa. These materials are obtained from synthetic or natural compound libraries and the methods to obtain or construct libraries are known in the art. For example, synthetic chemical library may be obtained from Maybridge Chemical Co.(UK), Comgenex(USA), Brandon Asociates(USA), Microsource(USA) and Sigma-Aldrich(USA). The chemical library of natural origin may be obtained from Pan Laboratories (USA) and MycoSearch(USA). Further test substances may be obtained by various combinatorial library construction methods known in the art including for example, biological libraries, spatially addressable parallel solid phase or solution phase libraries, synthetic libraries that require deconvolution, “1-bead 1-compound” libraries, synthetic libraries that require that use affinity chromatography. Various library preparation methods may be found in DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994, Houghten, R. A. et al. 1991, Nature 354:84-86 and the like.

In one embodiment, small molecules are used to identify agents useful for treating and/or preventing sepsis or symptoms associated therewith. For example small molecules with molecular weight of about less than 1,000Da such as 400 Da, 600 Da, or 800Da. If desired, small molecules may form part of a library, the total number of small molecules included therein may vary from dozens to millions. Test substance of a library may be composed of peptides, peptoides, circular or liner oligomeric compounds, template based compounds such as benzodiazepine, hydantoin, biaryls, carbocyclic and polycyclic compounds such as naphthalene, phenothiazine, acridine, steroids and the like, carbohydrate and amino acid derivatives, dihydropyridine, benzhydryl and heterocyclic compounds such as triazine, indole, thiazolidine and the like, but does not limited thereto.

In one embodiment, small molecules which is able to particularly suppressing or inhibiting the phosphorylation and/or kinase activity of RIP2 are employed. Also kinase inhibitors particularly inhibitors of tyrosine kinases or phosphorylation inhibitors which are known in the art may also be used as test compounds.

Also employed for the present methods are biologics. Biologics generally refers to cells or biomolecules such as proteins including antibodies, peptides or other proteins found in plasma, nucleic acids such as polynucleotides, carbohydrates, lipids or any materials produced in vivo or in vitro by biological systems such as cell culture system. For the purpose of the present invention, biomolecules may be used alone or in combination with others.

In other aspect, the present disclosure is directed to a pharmaceutical composition treating or preventing sepsis comprising as an effective ingredient inhibitors of NOD2 mediated pathway, particularly NOD2 and/or RIP2 inhibitors. The present disclosure is directed to modulating/inhibiting/suppressing NOD2 signal transduction pathway by targeting RIP2. Thus various materials known in the art that interfere with the NOD2 signaling pathway by inhibiting the expression and/or activities of NOD2 and/or expression, phosphorylation and/or kinase activity of RIP2 may be used for the present invention. Such inhibitors may be selected by the present screening methods.

The present inhibitors that suppress the expression and/or activities of NOD2 and/or the expression, phosphorylation and/or kinase activity of RIP2 may include small molecules, nucleic acids such as antisense oligonucleotides, siRNA, shRNA or miRNA or any combination thereof. In one embodiment, RIP2 inhibitors are small molecules for example such as Imatinib, Dasatinib, Niolotinib, Gefitinib, Erlotinib, Afatinib, Dacomitinib, Crizotinib, Sorafenib, Sunitinib, Pazopanib, Axitinib, Lapatinib, Vemurafenib, Everolimus, Temsirolimus, Dovitinib, or SB203580 and derivatives or analogs thereof, but does not limited thereto. In one particular embodiment, tyrosine kinase inhibitors such as SB203580 are used to suppress the kinase activity of RIP2.

The term expression refers to a process where proteins are synthesized in cells or in vitro and includes transcription from a gene to mRNA and translation from mRNA to protein.

The NOD2 or RIP2 inhibitors of the present disclosure specifically bind or recognize NOD2 or RIP2 and interfere with their biological function in cells or in vitro. In one embodiment, the inhibitors are antibodies which are able to interfere with phosphorylation and/or kinase activity of RIP2. Various antibodies or any equivalent thereof may be used for the present invention and include whole antibodies and antigen binding fragments. Also antibodies may be of IgG, IgM, IgD, IgE, IgA or IgY type, or belong to IgG1, IgG2, IgG3, IgG4, IgA1 or IgA2 class or subclasses thereof. Also used for the present invention are monoclonal, polyclonal, chimeric, single chain, bispecific, or humanized antibodies.

In other embodiment, the inhibitors are polypeptides which are able to interfere with phosphorylation and/or kinase activity of RIP2. The term polypeptides refer to naturally occurring or synthetic polymer of amino acids and may include oligonucleotides and peptides with various lengths. In one embodiment, the polypeptides encompass all or part of a region of RIP2 that has a kinase activity and work as a competitive inhibitor by interfering with RIP. Further unmodified or modified peptides for example glycosylation, acetylation, phosphorylation and the like may also be used.

The composition of the present disclosure may further comprise one or more effective ingredient with activities identical or similar to the NOD2 or RIP2 inhibitors of the present disclosure and/or agents that enhance the availability of the present inhibitors in vivo. Also encompassed are the combined use of the present inhibitors with other therapies to treat and/or prevent sepsis, which includes surgery, drug therapy and use of biological response modifiers. The present composition may further includes one or more pharmaceutically acceptable carriers, which includes but does not limited to, saline, sterilized water, Ringer's solution, buffered saline, dextrose solution, maltodextrin solution, glycerol, ethanol, liposome. If desired, the composition may further include antioxidant, buffer, antibacterial agents, and other additives known in the art to prepare pharmaceutical compositions. The present composition may be formulated into injectable formulations or oral formulations such as capsules, granules, or tablets by methods known in the art using one or more of diluents, dispersing agents, surfactants, binders and lubricants. Also encompassed for the present invention is a target specific composition combined with an antibody or other ligands that specifically recognize a molecule present on a target tissue or organ of interest. Further latest edition of Remington's Pharmaceutical Science (Mack Publishing Company, Easton Pa.) may be referred for the preparation and formulation of pharmaceutical composition.

The present composition can be administered by various routes known in the art such as oral or parenteral delivery for example intravenous, subcutaneous, or intraperitoneal injections or delivery through patch, nasal or respiratory patches. In one embodiment, injections are preferred. Desirable or optimal dosage may vary among patients depending on various factors such as body weight, age, sex, general condition of health, diet, severity of diseases, and excretion rate. Dosages used for known kinase inhibitors of RIP2 may be referred. Where siRNA, miRNA, antisense oligonucleotides, shRNA and proteins including polypeptides are used, parenteral deliveries are preferred. The typical unit dosage includes but does not limit to for example about 0.01 mg to 100 mg a day. Typical daily dosage ranges from about 1 μg to 10 g and may be administered one or multiple times a day.

Also embodied in the present disclosure is a method of treating sepsis by administering an effective amount of one or more of therapeutic agents that regulates NOD2-mediated pathway.

In one aspect, the present invention is directed to a method treating sepsis comprising administering to a subject in need thereof an effective amount of an inhibitor of the kinase activity of a RIP2 protein and/or an inhibitor of the phosphorylation of a RIP2 protein. In one embodiment, therapeutic agents screened by the present method or RIP2 inhibitors known in the art are used, which for example includes Imatinib, Dasatinib, Niolotinib, Gefitinib, Erlotinib, Afatinib, Dacomitinib, Crizotinib, Sorafenib, Sunitinib, Pazopanib, Axitinib, Lapatinib, Vemurafenib, Everolimus, Temsirolimus, Dovitinib and SB203580, but does not limited thereto.

As used herein, the term “therapeutically effective amount” or “effective amount” refers to the amount of a therapy, which is sufficient to treat, attenuate, reduce the severity of sepsis, reduce the duration of sepsis, prevent the advancement of sepsis, cause regression of sepsis, ameliorate one or more symptoms associated with sepsis, or enhance or improve the therapeutic effect(s) of another therapy.

In still other aspect, the present invention is directed to a method treating sepsis comprising administering to a subject in need thereof an effective amount of one or more NOD2 inhibitors and/or RIP2 inhibitors. In one embodiment, the inhibitors may be derived from small molecules, antibodies, antisense oligonucleotides, siRNAs, shRNAs miRNAs and polypeptides.

The present disclosure is further explained in more detail with reference to the following examples. These examples, however, should not be interpreted as limiting the scope of the present invention in any manner.

### EXAMPLES

### Materials and Methods

### 1. Mice and CLP-Induced Sepsis Model

Seven to 8-week-old WT C57BL/6 (B6) mice were purchased from the Orient Company Ltd (Seoul, Korea). Nod2^{-/-} mice on a B6 background were purchased from the Jackson Laboratory (Bar Harbor, Me., USA). Mice were bred and maintained under specific pathogen-free conditions at the Biomedical Research Institute Seoul National University Hospital. All animal experiments were approved by the Institutional Animal Care and Use Committee of CRISNUH. To perform CLP-induced sepsis, anesthesia was induced in mice by intraperitoneal (i.p.) administration of 2, 2, 2-tribromomethanol (Sigma-Aldrich, St. Louis, Mo., USA). The mouse cecum was exposed through a 1 cm incision, and the cecum was ligated below the ileocecal valve with a 5-0 Ethilon suture (Ethicon, Somerville, N.J., USA) without causing a bowel obstruction. The cecum was then punctured with a 26-gauge needle. Survival rates were determined over a 10-day period, with assessments every 12 h.

2. Injection of Mice with Recombinant Proteins and mAbs

To deplete Gr-1⁺cells in vivo, WT B6 or NOD2−/− mice were i.p. injected with anti-Gr-1 mAb (160 μg/injection) (BD Bioscience, Sparks, Md., USA) 2 and 3 days prior to CLP. Recombinant mouse (mr) IL-1β (40 μg/mouse) or IL-10 (30 μg/mouse) (ProSpec-Tany TechnoGene, Rehovot, Israel) was i.p. injected into WT or Nod2^{-/-} mice 4 or 12 h after CLP, respectively. mrC5a (5 μg/injection) or anti-05a mAb (100 μg/injection) (R&D Systems Inc., Minneapolis, Minn., USA) was i.p. injected into WT or Nod2^{-/-} mice 4 and 12 h after CLP.

### 3. Measurement of CFUs

Bacterial CFUs were determined by plating serial dilutions of blood and liver homogenates onto blood agar plates (Hanil Komed, Seoul, Korea), which were incubated in 5% CO₂ at 37° C. overnight. The number of colonies was counted 18 h later.

### 4. Cell Preparation and Culture

Peritoneal cell were collected from peritoneal fluid of WT B6 mice. Cells (5×10⁵) were cultured with MDP, LTA, or LPS (all from Sigma Aldrich) in the presence or absence of MDP (20 μg/ml) for 20-24 h. SB203580 kinase inhibitors were used in the concentration of 20 nM for cell culture and 50 μg/mouse for i.p. injection.

### 5. Enzyme-Linked Immunosorbent Assay (ELISA)

Concentrations of IL-1β, IL-6, IL-10, IFN-γ, TNF-α and C5a in serum and peritoneal fluid were measured in CLP-induced sepsis mice. All cytokines were measured using a BD Bioscience ELISA kit according to the manufacturer's instructions. Colorimetric reactions were stopped by adding 3 N HCl, and the optical absorbance at 450 and 570 nm was determined using a spectrophotometer.

### 6. Flow Cytometric Analysis

PLF cells were incubated with antibodies on ice for 30 min in 100 μl of staining buffer (0.5% BSA). FITC-conjugated anti-Ly-6G, phycoerythrin (PE)-conjugated anti-CD4 mAb, PE-conjugated anti-CD8, PE-conjugated anti-MHC class II, FITC-conjugated anti-CD80, FITC-conjugated anti-CD86, FITC-conjugated anti-annexin V mAbs, and 7AAD (7-amino-actinomycin D) were purchased from BD Biosciences. PE Cy5- or Alexa 647 (eBioscience, San Diego, Calif., USA)-conjugated anti-F4/80 mAb was used. Stained cells were then run on an LSR II and FACs caliber (BD Bioscience) and analyzed using Flowjo software (Treestar, Ashland, Oreg., USA).

### 7. Sorting Peritoneal Fluid Cells

Peritoneal fluid cells from WT B6 and Nod2^{-/-} mice with CLP were obtained to isolate Ly-6G⁺F4/80⁻ and Ly-6G⁻F480⁺ cells. The peritoneal fluid cells were stained with FITC-conjugated anti-Ly-6G mAb (BD Biosciences) and PE Cy5-conjugated anti-F4/80 mAb (eBioscience). Stained cells were then sorted on a BD FACSAria flow cytometer (Franklin Lakes, N.J., USA). Sorted Ly-6G⁺F4/80⁻ and Ly-6G⁻F480⁺ cells were isolated at 98% purity.

### 8. Real-Time PCR Analysis

An RNeasy Mini kit (Qiagen, Courtaboeuf, France) was used to isolate mRNA from sorted peritoneal fluid Ly-6G⁺F4/80⁻ and Ly-6G⁻F480⁺ cells. RNA (3 □g) was reverse-transcribed into cDNA using M-MLV Reverse Transcriptase (Promega, Madison, Wis., USA). PCR was performed using cDNA as a template with primers and probes from Applied Biosystems (Foster City, Calif., USA) and Biosource (Camarillo, Calif., USA) for GAPDH, NOD2, IL-1β and IL-10 (TaqMan predeveloped Assay Reagent). The results of the gene expression analysis were normalized with respect to GAPDH expression.

### 9. Measurement of Bacterial Phagocytosis by Peritoneal Immune Cells

Peritoneal fluid cells of WT B6 or Nod2^{-/-} mice were cultured with FITC-labeled E. coli (Invitrogen, Carlsbad, Calif., USA) for 15 min. Cells attached on the plates were washed with warm PBS three times and then treated with 0.2% trypan blue for 1 min at room temperature. Cells were fixed with 4% formalin for 15 min and cultured with FITC-labeled E. coli. to estimate nonspecific binding of E. coli on the cell surface. These cells, which were cultured with FITC-labeled E. coli, were then run on the FACs caliber machine and analyzed with Flowjo software.

### 10. Statistical Analysis

Survival data were plotted as Kaplan-Meier survival curves and analyzed using the log-rank test. Statistical significance was analyzed using Prism 5.0 (GraphPad Software Inc, San Diego, Calif., USA). One-way and two-way analyses of variance (ANOVA) and t-tests was performed, and a post hoc test was used if P<0.05. Data are expressed as the mean±standard error of the mean.

### Example 1

### NOD2-Mediated Signals Promote Polymicrobial Sepsis by Enhancing C5a Generation

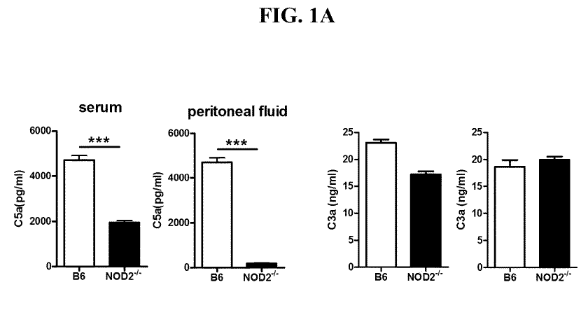

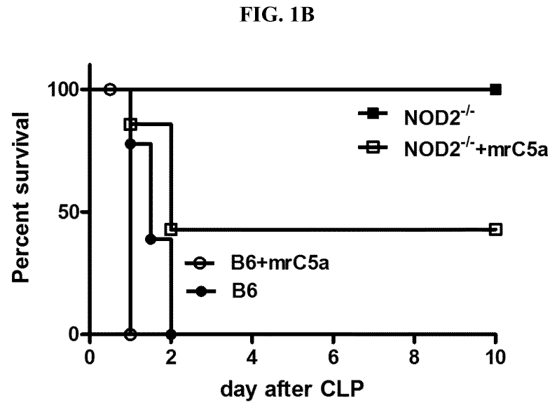

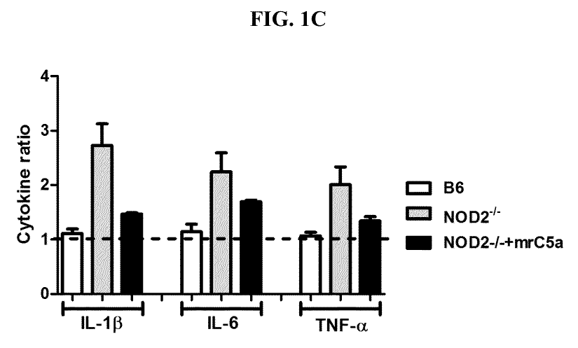

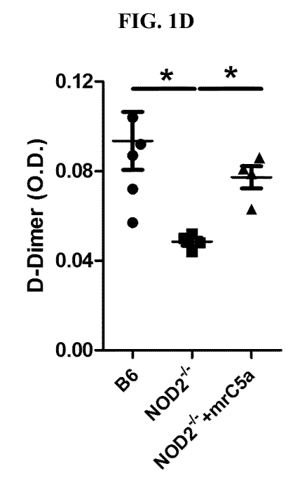

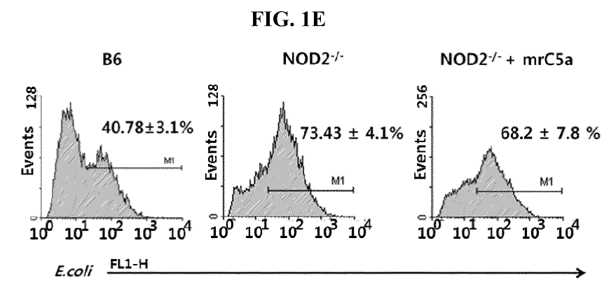

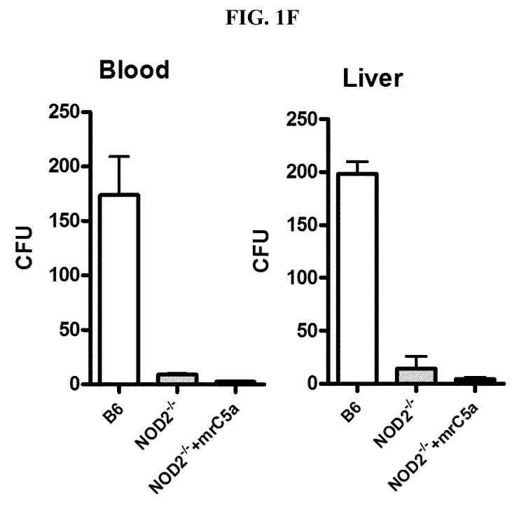

To investigate whether NOD2 regulates complement generation during sepsis, Cecal ligation and puncture (CLP) in wild-type (WT) and Nod2^{-/-} mice were performed. Results are shown in FIG. 1. During polymicrobial infection, serum and peritoneal C5a levels were higher in Nod2^{-/-} mice than in WT B6 mice, whereas C3a levels were similar between two mouse groups (FIG. 1A). All Nod2^{-/-} mice were alive up to 10 days after CLP, whereas all WT B6 mice were dead within 2 days. Moreover, injection of Nod2^{-/-} mice with recombinant C5a decreased survival rates during polymicrobial infection, whereas treatment of WT mice with recombinant C5a did not cause survival changes (FIG. 1B). These findings suggest that NOD2-mediated C5a generation contributes to development and severity of sepsis. To rule out a possibility that difference in the cecal bacterial composition of WT B6 and Nod2^{-/-} mice affects CLP-induced sepsis, cecal contents obtained from WT B6 or Nod2^{-/-} mice were injected i.p. into WT B6 and Nod2^{-/-} mice after their cecums were ligated but not punctured. Consistent with our results in CLP-induced sepsis, the survival rates after the injection of Nod2^{-/-} or WT B6 cecal contents were higher in the WT B6 mice than Nod2^{-/-} mice (Data not shown), indicating that differences in the intestinal bacterial profile of WT and Nod2^{-/-} mice constitute only a minimal contribution to NOD2-mediated regulation in sepsis. Collectively, these findings suggest that NOD2-mediated signals enhance C5a, but not C3a generation, thereby promotes sepsis.

During bacterial septic shock, C5a triggers coagulopathy and neutrophil dysfunction, thereby defective bacterial clearance and cytokine production (Ward, P. A. The dark side of C5a in sepsis. Nat Rev Immunol 4, 133-142 (2004)). Therefore, to estimate C5a effects on immune responses in WT B6 and Nod2^{-/-} mice during infection, we examined the responsiveness of immune cells against lipopolysaccharide (LPS) and macrophage phagocytic activity, bacteria colony-forming units (CFUs), and levels of serum D-dimer after CLP. To estimate the responsiveness of peritoneal immune cells against LPS, peritoneal cells obtained from B6 or Nod2^{-/-} mice 24 h after CLP were stimulated with LPS or PBS (FIG. 1C). The responsiveness of peritoneal immune cells against LPS was determined by estimating ratios of individual cytokines produced against LPS versus PBS. Peritoneal immune cells obtained from Nod2^{-/-} mice 24 h after CLP produced higher IL-1β, IL-6, and TNF-αlevels against LPS than did WT peritoneal cells. The levels of serum D-dimer in Nod2^{-/-} mice were lower than those in WT mice (FIG. 1D). However, peritoneal cells obtained from Nod2^{-/-} mice engulfed more FITC-conjugated Escherichia coli than did WT peritoneal cells (FIG. 1E). Consistent with these findings, bacteria CFU levels in the blood and liver homogenates were higher in WT B6 mice than in Nod2^{-/-} mice (FIG. 1F). Recombinant C5a administration to Nod2^{-/-} mice with CLP reversed cytokine production by peritoneal immune cells and serum D-dimer levels, but not phagocytosis activity and bacterial CFUs. These findings suggest that NOD2-mediated signals triggers dysfunction of immune cells in terms of cytokine production and coagulopathy by enhancing C5a levels, whereas NOD2-mediated immune response regulates bacterial phagocytic activity and CFU levels during polymicrobial infection in a C5a-independent manner.

### Example 2

### NOD2-Mediated Signals Produce IL-1β and IL-10 Production by Ly6-G⁺ Granulocytes During Sepsis

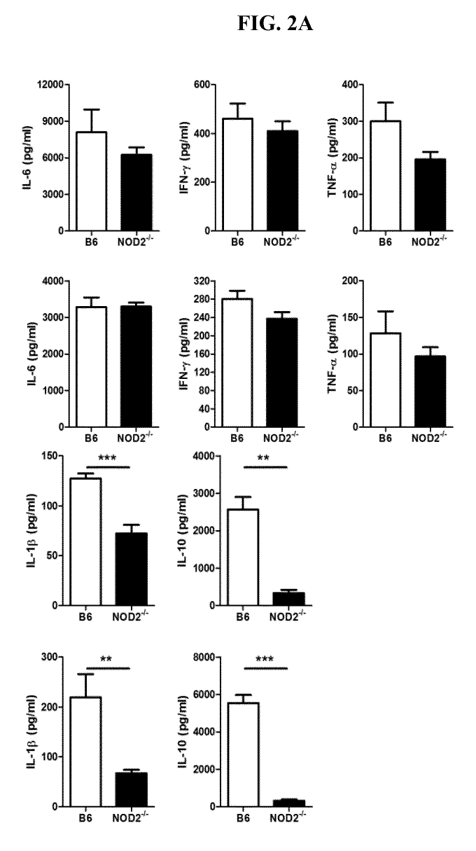

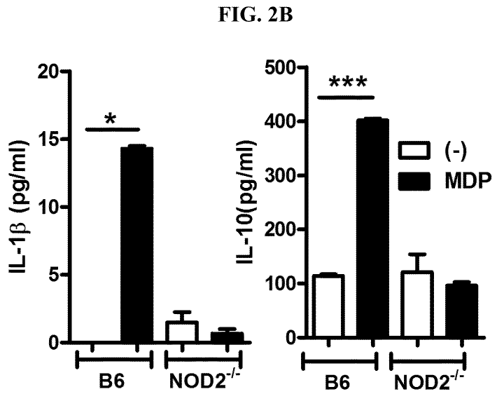

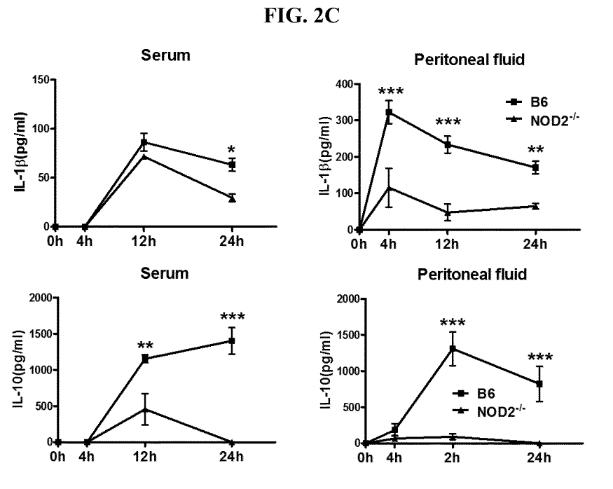

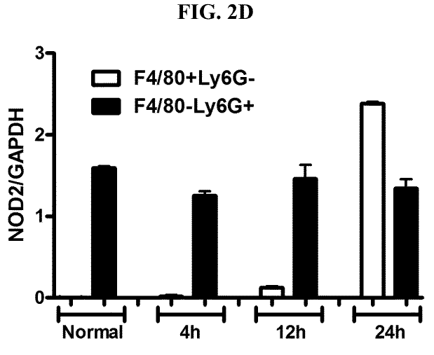

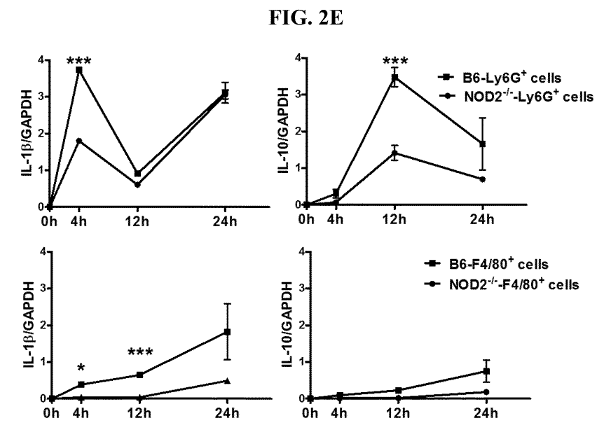

To investigate the mechanism by which NOD2 enhances C5 generation during polymicrobial infection, serum and peritoneal levels of various cytokines in WT and Nod2^{-/-} mice were estimated after CLP. Among the cytokines tested, serum and peritoneal IL-1β and IL-10 levels of WT mice were significantly higher than those of Nod2^{-/-} mice, whereas IL-6, TNF-α, and IFN-γ levels in WT mice were similar to those in Nod2^{-/-} mice (FIG. 2A). To confirm whether NOD2-mediated signals induce IL-1β and IL-10 production by immune cells, peritoneal cells from WT and Nod2^{-/-} mice were cultured with MDP, a NOD2 agonist. Upon MDP treatment, WT peritoneal immune cells produced IL-1β and IL-10, whereas NOD2-deficient cells minimally produced both IL-1β and IL-10, indicating that NOD2-mediated signals induce IL-1β and IL-10 production by peritoneal immune cells during sepsis (FIG. 2B). A kinetic analysis revealed that IL-1β and IL-10 levels in peritoneal fluid peaked 4 and 12 h after CLP, respectively, and then decreased gradually (FIG. 2C). Serum IL-1β levels peaked 12 h after CLP and were significantly higher in WT mice as compared with Nod2^{-/-} mice at 24 h, whereas serum IL-10 levels in WT increased continuously from 4 to 24 h after CLP, which were significantly higher than those of Nod2^{-/-} mice. Among immune cells, monocytes, granulocytes, and dendritic cells, but not T or B cells, express NOD 2 (Gutierrez, O. et al. Induction of Nod2 in myelomonocytic and intestinal epithelial cells via nuclear factor-kappa B activation. J Biol Chem 277, 41701-41705 (2002)).

Based on these findings, NOD2 expression was investigated in sorted F4/80⁺Ly-6G⁻ and F4/80⁻Ly-6G⁺ peritoneal cells of WT mice with CLP (FIG. 2D), to determine which cell types of peritoneal immune cells produce IL-10 and IL-1β during polymicrobial infection. Real-time PCR revealed that NOD2 expression in F4/80⁻Ly-6G⁺ cells was constitutive and sustained for 4, 12, and 24 h after CLP, whereas F4/80⁺Ly-6G⁻ cells minimally expressed NOD2 before and 4 and 12 h after CLP, but highly expressed NOD2 24 h after CLP. These findings indicate that F4/80⁻Ly-6G⁺ rather than F4/80⁺Ly-6G⁻ peritoneal cells predominantly express NOD2 at the early- and mid-time points of polymicrobial infection. Consistent with the kinetics of the IL-1β and IL-10 levels in serum and peritoneal fluid, F4/80⁻Ly-6G⁺ peritoneal cells from WT mice produced high IL-1β levels at 4 and 24 h, but low levels at 12 h. In contrast, F4/80⁺Ly-6G peritoneal cells produced high levels of IL-1β 24 h after CLP. Unlike IL-1β, F4/80⁻Ly-6G⁺ peritoneal cells from WT mice predominantly produced IL-10 12 h after CLP. Although the kinetics of IL-1β and IL-10 production were similar between Nod2^{-/-} and WT mice, individual cytokine levels were much lower in Nod2^{-/-} mice as compared with WT mice. These findings suggest that Ly-6G⁺ peritoneal cells rather than F4/80⁺ macrophages produce IL-1β and IL-10 during sepsis at different time points.

### Example 3

### NOD2-mediated IL-1β-dependent IL-10 Production by Ly6-G⁺ Granulocytes Enhances C5a Generation During Sepsis, while NOD2-Mediated IL-1□ Decreases Phagocytosis During Sepsis in an IL-10- and C5α-Independent Manners

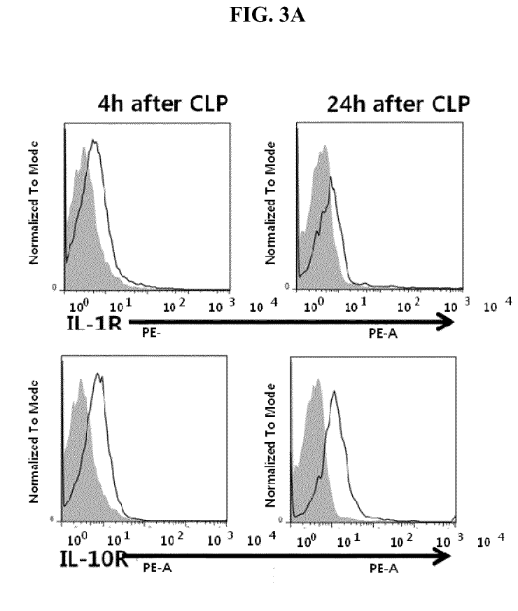

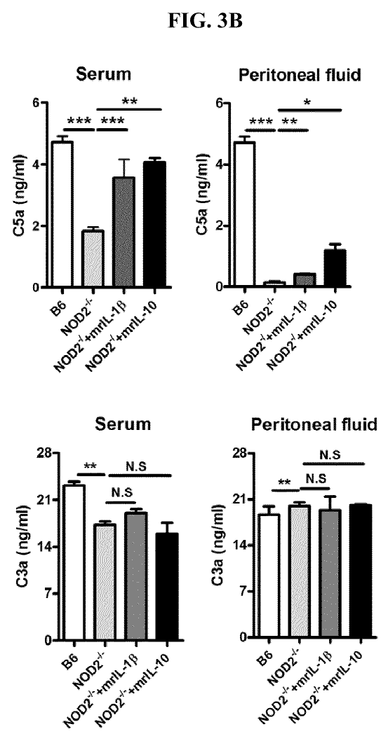

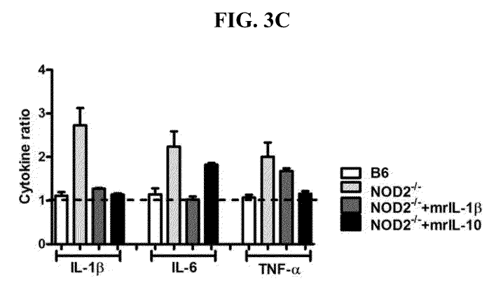

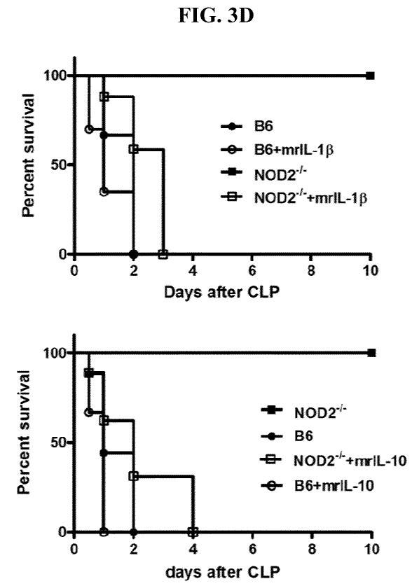

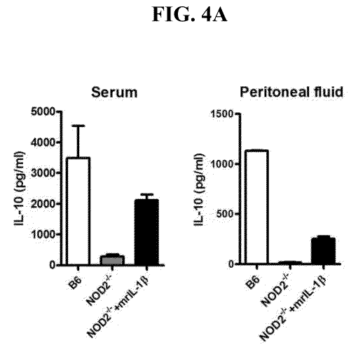

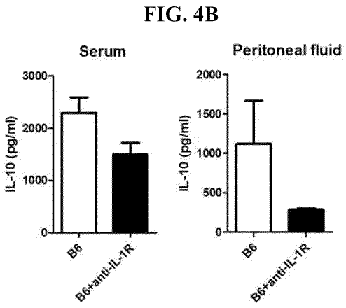

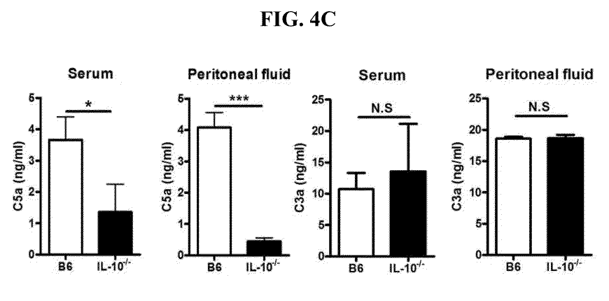

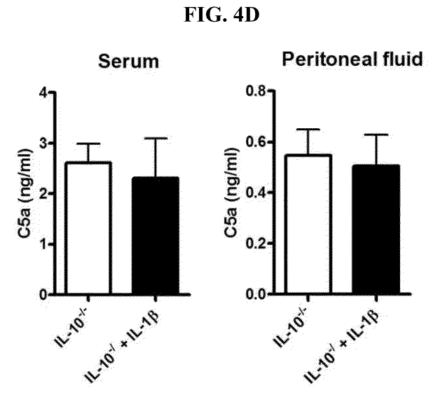

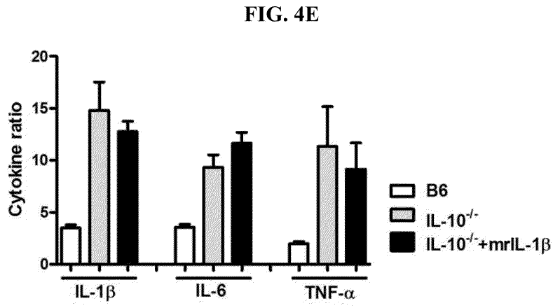

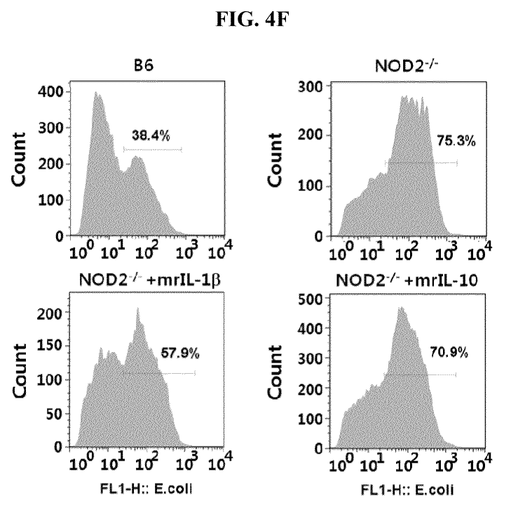

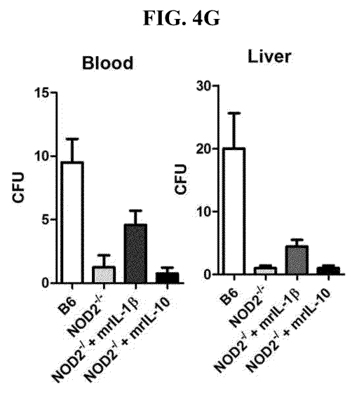

To investigate whether NOD2-mediated IL-1β and IL-10 production plays critical roles in the regulation of C5a generation during polymicrobial infection, we measured the expression of IL-1β and IL-10 receptors on peritoneal immune cells of WT mice and administered recombinant IL-1β or IL-10 into WT or Nod2^{-/-} mice 4 h or 12 h after CLP, respectively. The time points when the mice were injected with recombinant IL-1β or IL-10 were determined based on the kinetics of these cytokines in WT B6 mice during polymicrobial infection. Both IL-1β and IL-10 receptors were expressed on peritoneal cells of WT B6 mice with CLP (FIG. 3A). Administration of recombinant IL-1β or IL-10 enhanced serum and peritoneal C5a, but not C3a generation (FIG. 3B). Consistent with these results, recombinant IL-1β and IL-10 also suppressed LPS-mediated cytokine production by peritoneal immune cells obtained from Nod2^{-/-} mice with CLP (FIG. 3C). Furthermore, injection of recombinant IL-1β or IL-10 into Nod2^{-/-} mice reduced survival rates during CLP-induced sepsis, whereas these cytokine did not affect survivals of WT mice (FIG. 3D). These findings indicate that NOD2-mediated IL-1β and IL-10 production by Ly6-G⁺ granulocytes contributes to pathogenesis of polymicrobial sepsis by enhancing C5a generation. The IL-1β autocrine loop amplifies NOD2-mediated induction of pro- and anti-inflammatory cytokines in human monocyte-derived macrophages (Hedl, M. & Abraham, C. Distinct roles for Nod2 protein and autocrine interleukin-1beta in muramyl dipeptide-induced mitogen-activated protein kinase activation and cytokine secretion in human macrophages. J Biol Chem 286, 26440-26449 (2011)), which led us to hypothesize that IL-1β-dependent IL-10 production by Ly6-G⁺ cells may occur in NOD2-mediated immune response during polymicrobial infection. Recombinant IL-1β administration into Nod2^{-/-} mice enhanced serum and peritoneal IL-10 levels during polymicrobial infection, while anti-IL-1R mAb reduced IL-10 levels in WT B6 mice with CLP (FIGS. 4A and B), indicating that IL-1β-dependent IL-10 production occurred in NOD2-mediated sepsis.

Next, to explore whether IL-10 production regulates serum and peritoneal C3a and C5a levels during sepsis, we measured those levels in Il-10^{-/-} mice with CLP (FIG. 4C). Il-10^{-/-} mice showed C5a, but not C3a levels during polymicrobial infection, which was not altered by administering recombinant IL-1β (FIGS. 4C and D). Moreover, recombinant IL-1β did not alter LPS-induced cytokine production by peritoneal immune cells from Il-10^{-/-} mice with CLP. These findings suggest that a NOD2-mediated IL-1β-dependent IL-10 production by Ly6-G⁺ granulocytes regulate C5a generation during polymicrobial infection. Meanwhile, recombinant IL-1β but not recombinant IL-10, reduced phagocytosis of bacteria by peritoneal cells and bacterial CFUs in the blood or liver homogenates from Nod2^{-/-} mice with sepsis (FIG. 4F, G). These finding suggest that IL-1β not only deactivates immune cells via C5a generation, depending on IL-1β-dependent IL-10 production, but also reduces immune cell phagocytosis of bacteria in an IL-10 and C5a-independent manner during NOD2-mediated regulation of sepsis.

### Example 4

### NOD2-Mediated IL-1β-Dependent IL-10 Production Enhances C5a Generation by Suppressing CD55 Expression on Ly6-G⁺ Granulocytes

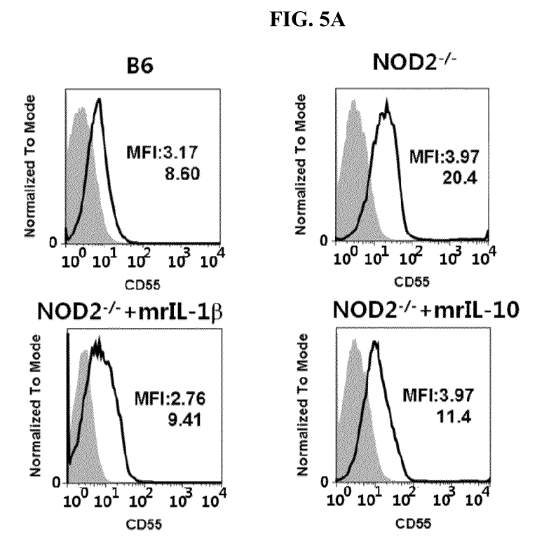

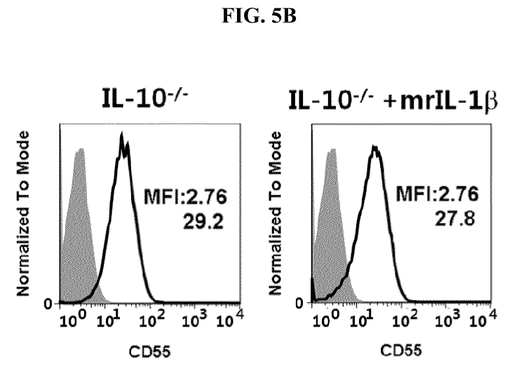

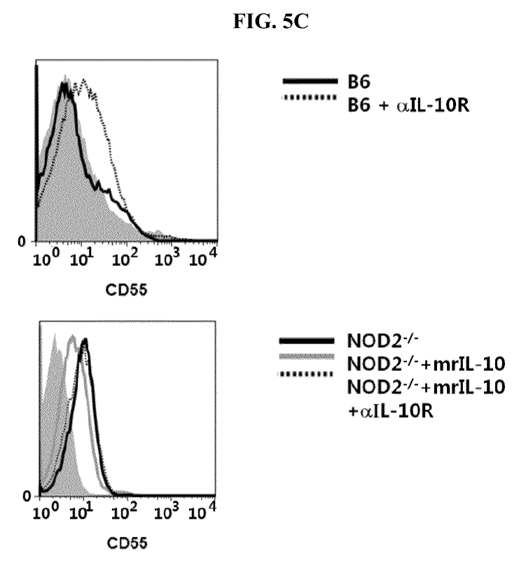

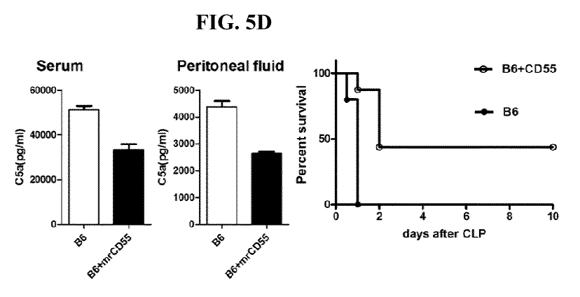

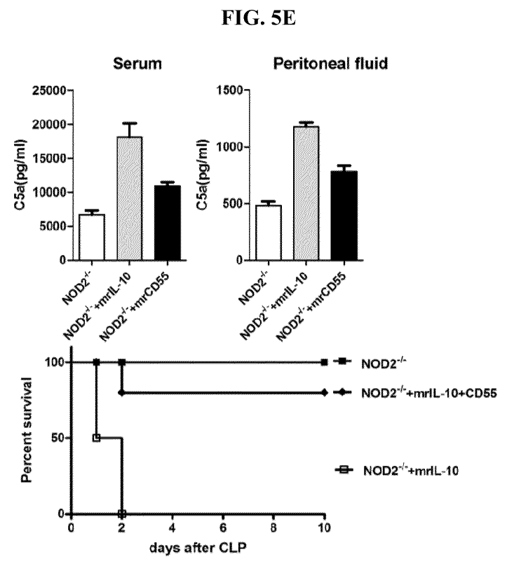

The processes of C5a generation in complement network are complicated. Nevertheless, several membrane molecules such as CD55 and CR1/2 on immune cells regulate complement network by inhibiting complement generation. Therefore, to functionally link expression of these molecules and C5a generation by NOD2-mediated IL-1β and IL-10 network during sepsis, CD55 and CR1/2 expression levels on Ly6-G⁺ peritoneal cells from WT B6 and Nod2^{-/-} mice were measured. CD55 expression levels on F4/80⁻Ly6-G⁺ peritoneal cells from Nod2^{-/-} and Il-10^{-/-} mice were higher than those of WT mice 24 h after CLP, whereas CR1/2 was not detected on F4/80⁻Ly6-G⁺ peritoneal cells from WT and Nod2^{-/-} mice (FIG. 5A). Moreover, recombinant IL-10 or IL-1β administration to Nod2^{-/-} mice decreased CD55 expression on F4/80⁻Ly6-G⁺ peritoneal cells during polymicrobial infection. However, IL-1β administration to Il-10^{-/-} mice did not decrease CD55 expression on F4/80⁻Ly6-G⁺ peritoneal cells (FIG. 5B). These findings suggest that NOD2-mediated IL-1β-dependent IL-10 production decrease CD55 expression on F4/80⁻Ly6-G⁺ peritoneal cells during polymicrobial infection.

Next, to explore whether CD55 expression on F4/80⁻Ly6-G⁺ peritoneal cells was dependent on IL-10 receptor engagement during septic shock, we administered anti-IL10 receptor mAb to WT B6 and Nod2^{-/-} mice given recombinant IL-10 during CLP. Anti-IL-10 receptor mAb increased CD55 expression on F4/80⁻Ly6-G⁺ peritoneal cells of WT B6 and Nod2^{-/-} mice given recombinant IL-10 (FIG. 5C). These findings suggest that IL-10 receptor engagement on F4/80⁻Ly6-G⁺ peritoneal cells decrease CD55 expression on cell surface, which regulates C5a generation during sepsis. To address this suggestion, peritoneal cells of WT B6 and Nod2^{-/-} mice with CLP were cultured and C5a amounts were measured in vitro. C5a generation was higher in culture fraction of WT peritoneal cells than that of NOD2-deficient cells, suggesting that reduced CD55 expression on WT Ly6-G⁺ granulocytes contributes to enhancement of C5a generation compared with high CD55 expressing NOD2-deficient peritoneal cells (FIG. 5D). To confirm this in vivo, soluble CD55 protein was administered to WT or Nod2^{-/-} mice given recombinant IL-10 during polymicrobial infection. As previously reported, soluble CD55 increased serum C5a levels in WT B6 mice. Furthermore, soluble CD55 protein decreased serum and peritoneal C5a levels in Nod2^{-/-} mice given recombinant IL-10, thereby showing low survival rates of these mice (FIGS. 5D and E). Taken together, NOD2-mediated IL-1β-dependent and/or -independent IL-10 production enhances C5a generation by suppressing CD55 expression on Ly6-G⁺cells, thereby aggravating polymicrobial sepsis.

### Example 5

### Blockade of NOD2 Signals Using SB203580 Attenuates Sepsis

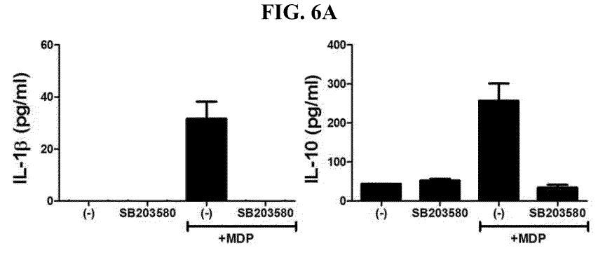

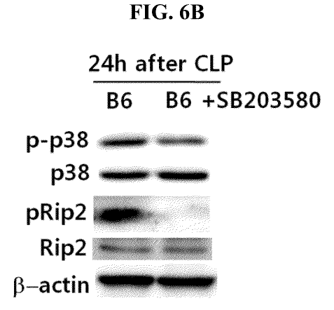

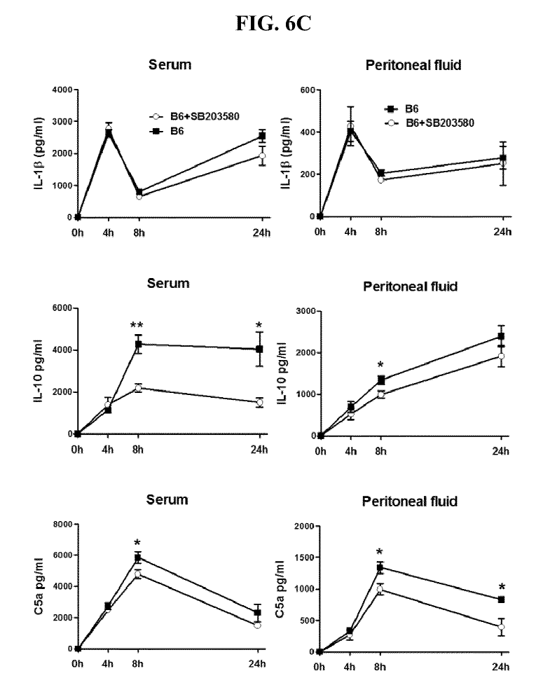

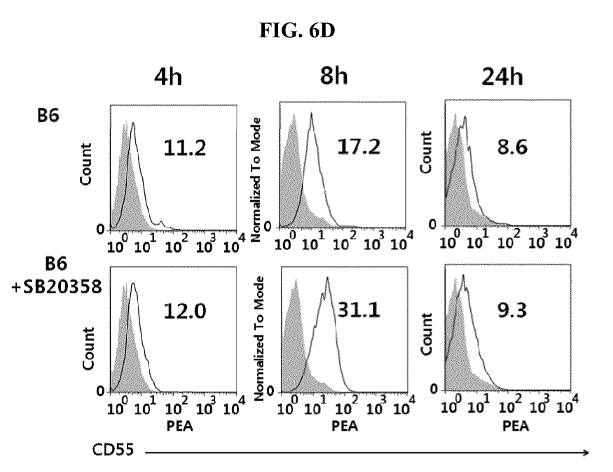

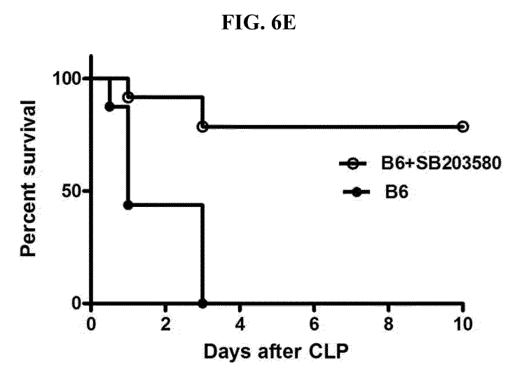

Upon activation, NOD2 oligomerizes and recruits the nuclear factor (NF)-κB-activating kinase receptor-interacting protein 2 (RIP2) via a homotypic CARD-CARD (caspase recruitment domain) interaction, triggering IκB phosphorylation and NF-κB activation. To confirm whether SB203580, an inhibitor for RIP2 and P38 inhibits NOD2-mediated IL-1β and IL-10 production, we cultured peritoneal immune cells of WT mice with SB203580 and/or MDP, and measured amounts of these cytokines. SB203580 decreased MDP-mediated IL-1β and IL-10 production by peritoneal cells, indicating that inhibition of NOD2-mediated signals suppresses IL-1β and IL-10 production by peritoneal cells during sepsis (FIG. 6A). To explore whether NOD2 signal blockade inhibits sepsis, we injected WT and NOD2 mice with SB203580. Nod2^{-/-} mice showed lower levels of RIP2 and P38 phosphorylation in peritoneal immune cells during CLP-induced sepsis than WT mice (FIG. 6B). Injection of SB203580 into WT mice reduced phosphorylation of RIP2 and P38 in peritoneal immune cells and levels of serum and peritoneal IL-1β, IL-10, and C5a during sepsis (FIGS. 6B and C). Moreover, administration of SB203580 to WT mice reduced CD55 expression levels of Ly6-G⁺ granulocytes, and increased survival rates during CLP-induced sepsis (FIGS. 6D and E). These findings suggest that NOD2 blockade using small molecule such as SB203580 inhibits C5a generation by enhancing CD55 on Ly6-G⁺ granulocytes, depending on IL-1β and IL-10 production by Ly6-G⁺ granulocytes, thereby increases survival rates of mice with sepsis. Based on these findings, we suggest that NOD2 may be a strong therapeutic target molecule for sepsis.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

What is claimed is:

1. A method of screening a therapeutic agent for sepsis, which comprises:
(i) providing a RIP2 protein;
(ii) contacting the RIP2 protein with a test substance wherein the substance is expected to inhibit the phosphorylation or kinase activity of the RIP2 protein; and
(iii) selecting a test substance as a candidate that decreases the phosphorylation level or the kinase activity of the RICK protein in comparison to a negative control that is not contacted with a test substance.

2. The method according to claim 1, wherein the RIP2 protein is provided as a cell.

3. The method according to claim 2, wherein the cell is a dendritic cell, a neutrophil, an epithelial cell, 293T, 293 or 293A or any combination thereof.

4. The method according to claim 1, further comprising selecting a test substance that increases the expression of a CD55 and/or decreases the level of C5a.

5. The method according to claim 1, wherein the test substance is derived from small molecules or biologics.

6. A method of screening a therapeutic agent for sepsis comprising:
(i) providing either a NOD2 or the CARD region of a NOD2 protein, and either a RIP2 or the CARD region of a RIP2 protein;
(ii) contacting the proteins with a test substance wherein the substance is expected to interfere with the interaction of the NOD2 or the CARD region of the NOD2 with the RIP2 or the CARD region of the RIP2 protein; and
(iii) detecting the interaction of (ii) and selecting a test substance as a candidate that interfere with the interaction.

7. The method according to claim 6, wherein the NOD2, the CARD region of the NOD2 protein, the RIP2 and the CARD region of the RIP2 protein are provided as cells.

8. The method according to claim 7, wherein the cells are a dendritic cell, a neutrophil, an epithelial cell, 293T, 293 or 293A or any combination thereof.

9. The method according to claim 7, further comprising selecting a test substance that increases the expression of a CD55 and/or decreases the level of C5a.

10. A method of treating sepsis comprising administering to a subject in need thereof an effective amount of an inhibitor of the kinase activity of a RIP2 protein and/or an inhibitor of the phosphorylation of a RIP2 protein.

11. The method according to claim 10, wherein the inhibitor of kinase activity is selected from the group consisting of Imatinib, Dasatinib, Niolotinib, Gefitinib, Erlotinib, Afatinib, Dacomitinib, Crizotinib, Sorafenib, Sunitinib, Pazopanib, Axitinib, Lapatinib, Vemurafenib, Everolimus, Temsirolimus, Dovitinib and SB203580.

12. A method of treating sepsis comprising administering to a subject in need thereof an effective amount of one or more NOD2 inhibitors.

13. The method according to claim 12, wherein the NOD2 inhibitor suppresses the expression or activity of the NOD2, which includes small molecules, antibodies, antisense oligonucleotides, siRNAs, shRNAs miRNAs and polypeptides.

14. A method of treating sepsis comprising administering to a subject in need thereof an effective amount of one or more RIP2 inhibitors.

15. The method according to claim 14, wherein the RIP2 inhibitor inhibit the expression or activity of the RIP2, which includes small molecules, antibodies, antisense oligonucleotides, siRNAs, shRNAs miRNAs and polypeptides.

16. A method of treating sepsis comprising administering to a subject in need thereof an effective amount of one or more inhibitors that interfere with RIP2 and NOD2 interaction.
